(19) United States
(12) Patent Application Publication
(10) Pub. No.: US 2012/0197358 A1
(43) Pub. Date: Aug. 2, 2012

(54) Light Therapy for Treating or Managing Diabetes and Metabolic Syndrome

(76) Inventor: Marvin A. Prescott, Los Angeles, CA (US)

(21) Appl. No.: 13/412,983

(22) Filed: Mar. 6, 2012

Related U.S. Application Data
(63) Continuation-in-part of application No. 13/079,627, filed on Apr. 4, 2011.
(60) Provisional application No. 61/322,748, filed on Apr. 9, 2010.

(51) Int. Cl. A61N 5/067; A61N 5/06

(52) U.S. Cl. 607/89; 607/88

(57) ABSTRACT

Techniques for managing diabetes and pre-diabetes using light are disclosed herein. In one example, a light generating device is positioned to a body part of a diabetic or pre-diabetic patient, and a beam of light generated by the light generating device is directed to the body part of the patient to control blood sugar level of the patient. In various embodiments, the beam of light directed to the patient can also help to control the blood lipid level such as triglyceride level, blood liver enzyme of the patient, and various other symptoms of diabetes and pre-diabetes. In various embodiments, the body part includes body area rich in adipose tissue, such as the abdominal area, thigh, buttocks, and upper arms of the patient.

### CROSS REFERENCE TO OTHER APPLICATIONS

This application is a continuation in part of co-pending U.S. patent application Ser. No. 13/079,627 (Attorney Docket No. DRP10301) entitled Method of Managing Metabolic Syndrome filed Feb. 4, 2011 which in turn claims priority from U.S. Provisional Patent Application No. 61/322,748 filed on Apr. 9, 2010, wherein the entirety of U.S. priority applications is herein incorporated herein by reference for all purposes.

### BACKGROUND OF THE INVENTION

Diabetes Mellitus, often referred to as diabetes, is a disorder in which blood glucose levels are abnormally high. Diabetes is caused by the body's failure to produce insulin (Type 1 diabetes) and/or inability to use insulin properly (Type 2 diabetes). It is estimated over 25 million Americans have diabetes and an additional 79 million Americans have pre-diabetes (or metabolic syndrome) for which blood glucose levels are higher than normal but not yet high enough to be diagnosed as diabetes.

Currently there is no cure for diabetes, patients are typically put on a life-long regimen of insulin and/or non-insulin medications aimed at managing blood sugar, which can be very inconvenient and often not very effective. Failure to properly maintain blood sugar levels often result in serious complications, including acute and often life threatening ketoacidosis and hyperosmolar (nonketotic) coma, particularly among Type 1 diabetic patients, and long-term complications such as heart attacks, strokes, diabetic retinopathy, kidney failure, and poor circulation of limbs that can lead to infections and amputations. Improved therapies for managing or treating diabetes and pre-diabetes are needed.

### BRIEF DESCRIPTION OF THE DRAWINGS

Various embodiments of the invention are disclosed in the following detailed description and the accompanying drawings.

FIG. 1 illustrates an example light-generating device for treating or managing diabetes and metabolic syndrome (or pre-diabetes).

FIG. 2 illustrates the inner electronic components of the light-generating device of FIG. 1.

FIG. 3 illustrates an example external light-generating device with contoured body configured to fit a patient's body contour.

FIG. 4 illustrates an example method for managing or treating diabetes and metabolic syndrome with a portable light generating device.

FIG. 5 illustrates an example embodiment for managing or treating diabetes and metabolic syndrome.

FIG. 6 illustrates an example embodiment for managing or treating diabetes and metabolic syndrome.

FIG. 7 illustrates another example embodiment for managing or treating diabetes and metabolic syndrome.

FIG. 8 illustrates another example embodiment for managing or treating diabetes and metabolic syndrome.

### DETAILED DESCRIPTION

The invention can be implemented in numerous ways, including as a method; a process; a therapy; an apparatus; a system; a device (external and/or implantable); and a composition of matters. A detailed description of one or more embodiments of the invention is provided below along with accompanying figures that illustrate the principles of the invention. The invention is described in connection with such embodiments, but the invention is not limited to any embodiment. In the specification, the various implementations of the invention may be referred to as techniques. In general, the order of the steps of disclosed processes may be altered and one or more steps of disclosed processes may be omitted within the scope of the invention.

Techniques for managing diabetes and pre-diabetes using light are disclosed herein. In various embodiments, a light generating device is positioned to a body part of a diabetic or pre-diabetic patient, and a beam of light generated by the light generating device is directed to the body part of the patient to control blood sugar level of the patient. In various embodiments, the beam of light directed to the patient can also help to control the blood lipid level such as triglyceride level, blood liver enzyme of the patient, and various other symptoms of diabetes and pre-diabetes. In various embodiments, the body part includes body area rich in adipose tissue, such as the abdominal area, thigh, buttocks, and upper arms of the patient. In various embodiments, the body part is adjacent to the pancreas of the patient. In various embodiments, the light beam has one or more wavelength in the range of 400 to 1300 nm. In various embodiments, the light beam has wavelength in the red and near infrared region. Light in this wavelength are known to penetrate tissue well and free from carcinogenic effect of lights in other wavelength region. In various embodiments the light beam has a wavelength in the range of 610 to 900 nm. In various embodiments, the light beam has a wavelength in the range of 800 to 900 nm. In various embodiments, the light beam is a low intensity light beam generated by for example a power of 1-12 mW using for example a laser diode or light emitting diode. It is conceivable a lower intensity light beam may be used, provided the light beam can penetrate the body to reach the targeted tissue and the exposure time is long enough that the patient receives adequate light therapy dose. It is also conceivable the light beam is a higher intensity light beam, provided that the exposure time is short and the patient is not over dosed. In various embodiments, the light beam is directed to a body part of the patient according to a prescribe regimen.

Clinical studies conducted on patients having metabolic syndrome (or pre-diabetes) and Type 1 and 2 diabetes, which will be discussed further below, show that low intensity light therapy described herein can decrease and stabilize blood sugar, lipid and liver enzyme levels of the patients, thus can be used to treat and manage diabetes and pre-diabetes. The inventor herein hypothesizes that low intensity light works by increasing nitric oxide (NO) production and bioavailability, which in turn increases adiponectin synthesis by the adipose tissues, which in turn sensitizes the body's response to insulin, which in turn helps to regulate blood glucose, triglyceride level, and lowering liver enzymes due to fatty liver disease caused by elevated triglyceride level in diabetic and pre-diabetic patients. In addition, nitric oxide is a powerful vasodilator and an increased nitric oxide level also increases blood flow to adipose tissues, which promotes normal cellular function including adiponectin synthesis and function and normal glucose metabolism.

Although the mechanisms of the light therapy need to be ascertained through further studies, however various existing researches on adiponectin, nitric oxide (NO) and light therapy support the above hypothesis. It is well known that for most patients having diabetes and pre-diabetes (or metabolic syndrome) the body does not produce enough insulin or is resistance to the effect of insulin or both. This causes glucose to build up in the blood stream since it cannot enter and be utilized by the cells efficiently. It is also known that adiponectin, a protein hormone produced and secreted exclusively by adipocytes (fat cells), modulates glucose and lipid metabolism by mimicking the effect of insulin, and a decrease in the circulating levels of adiponectin, caused by interactions of genetic factors and environmental factors, contribute to the development of metabolic syndrome and diabetes and a up-regulation of adiponectin suppresses symptoms of diabetes and metabolic syndrome. In addition, there have been some evidences showing that adiponectin synthesis is stimulated by nitric oxide (NO), an important cellular signaling molecule involved in many physiological and pathological processes and also an powerful vasodilator. It is further shown that nitric oxide (NO) increases adiponectin synthesis by stimulating mitochondrial biogenesis which in turn increases adiponectin synthesis in the adipose tissue (Koh et al., Endo April 2010 Vol. 298 No. 4 E846-E853). It has also been shown that low intensity light increases nitric oxide (NO) synthesis by cytochrome C oxidase and facilitates the release of biologically active nitric oxide (R.O. Poyton; Therapeutic Photomodulation: Nitric Oxide and a Novel Function of Mitochondrial Cytochrome C Oxidase. The study also shows that O₂ is increased by nitric oxide. Published online Feb. 20, 2011). It is further shown that low intensity light upregulates nitric oxide synthase (NOS) expression (Chen et al.; Lasers Surg Med. 2008 January: 40(1): 46-54).

In addition the inventor also hypothesizes that low intensity light therapy suppresses the hyperactive immune system which is present in a large portion of diabetic and pre-diabetic patients. This may keep the body from autoimmune destruction of beta pancreatic cells that are responsible for insulin production, particularly in Type 1 diabetes. Recent research suggests that the low intensity light therapy increases tissue oxygen by increasing nitric oxide synthase, intra-cellular and extra-cellular nitric oxide. The increased oxygen level may mobilize adipose mesenchymal stem cells (MSC) which are known to attract and inhibit the autoreactive T-cells function which are responsible for destruction of beta pancreatic cells particularly in Type 1 diabetes. The immunosuppressive effect of mesenchymal stem cells has been shown to occur mainly through the soluble factors by MSC. Among the possible mediators identified, indoleamine 2,3-dioxygenase (ID)), inducible nitric oxide synthase, and heme oxygenase-1 as well as the secretion of human leukocyte antigen G, transforming growth factor-beta, interleukin (IL)-6 or prostaglandin E2 have been postulated to play a role.

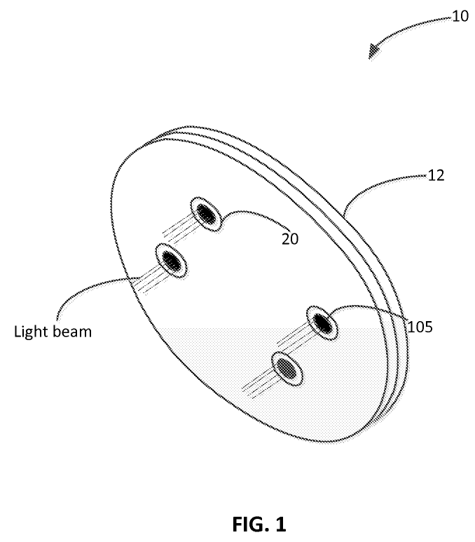

FIG. 1 illustrates an example light-generating device 10 for treating or managing diabetes and pre-diabetes. One or more light-generating devices 10 may be positioned to a patient's body, for example, to a specific body part or body region, and operated according to a prescribed light regimen. The specified body part or region may be a region that rich in underlying adipose tissue, such as stomach (visceral) areas, thigh areas, buttocks areas, upper arm areas. The light-generating devices 10 may be positioned to (e.g. attached via bandage, strap) the external surface of the skin of the specific body region or may be implanted to the specific body region, for example in the subcutaneous tissue of the specific body region.

Light-generating device 10 includes a body 12. The material used to form the body 12 may depend on whether the light generating device is configured as an external device or implantable device.

In various embodiments, the light generating device is an implantable device that is configured to be implanted inside a patient's body, such as in a subcutaneous space, and in such embodiments, the body should be made of biocompatible materials that can exist in harmony with the patient's body without causing deleterious changes when implanted. Important criteria of biocompatible material include for example that the material being nontoxic and eliciting little or no immune response from the patient's body. Example biocompatible materials that can be used to form the body include titanium metal and glass ceramics. In various embodiments, the device may be embedded in various scar prevention materials, such as collagen and microvascular construct (MVC) to minimize scar formation around the device when it is implanted. In various embodiments, the body may be coated with a layer of nanoporous ceramic membrane to prevent protein build up around the implanted device. The use of implantable light generating device for managing or treating diabetes or metabolic syndromes has the advantage of ensuring patient compliance and minimize or eliminate injuries, particularly retinal injuries caused by laser light if laser light is used as the light source.

In various embodiments, the light generating device is an external device. In various embodiments, the device may be a wearable and portable device that is configured to be positioned over the skin of a patient, and in such embodiments, the biocompatibility of the material for forming the body may not be important, so it can be made of a variety of materials as long as the material provides adequate protection to the various components of the light-generating device. For example, the light generating device can be made of p-cell or plastozote or lux plastic to make it non-flexible, light-weight, and portable. Alternatively, the body of the light-generating device may be made of flexible materials, such as molded silicone, to render the device flexible, light-weight, and portable. The use of portable external light generating device allows a patient to receive non-invasive light treatment for managing or treating diabetes or metabolic syndromes without frequent visit to the doctor's office. Alternatively, the device may not be a portable device, for example, the device may be placed in a caregiver's office (e.g., physician's office) and the patient may be required to travel to the caregiver's office to be treated to ensure proper use and safety.

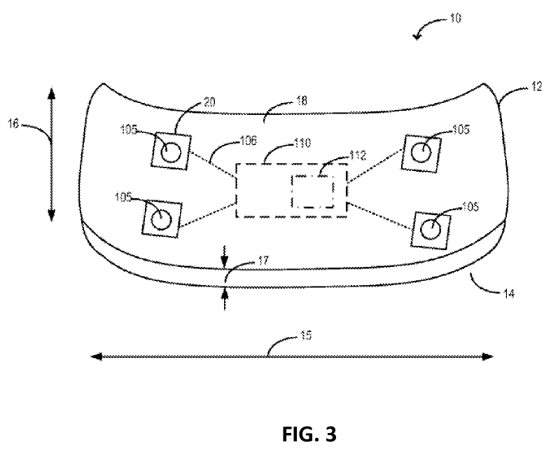

The body 12 may be designed as a casing within which are housed a light source, a power source, a programmable controller, and/or other sensors of the device 10. The device may be designed as virtually any shape. As a non-limiting example, the device 10 is depicted in FIG. 1 as having an oval body 12. In another non-limiting example, the device is substantially rectangular body 12 having a length 5 inches, a width 2.5 inches, and a thickness ¼ inch. The dimensions of the device may be adjusted based on the desired application of the device. When the device is configured as an external device, the body 12 may be contoured to reduce discomfort when the device is attached to the patient's body (as shown in FIG. 3). The contour 14 of the device may be adjusted to conform to the body part to which it is to be attached. For example, the device may be contoured differently if designed for use on the stomach or visceral adipose area than when designed for use on the thigh or upper leg area. Alternatively, the body of the device may be made of flexible material so the device can conform to the contour of the body part by virtue of its flexible nature.

The body 12 of the device may include openings 20, formed therein, to accommodate light produced by an underlying light source 105. The openings may be provided with clear windows to protect the underlying light source from dust, oils, dirt, etc. The clear windows may be formed of glass, plastic, titanium or other suitable materials. Alternatively, the clear windows may be formed of the same material as the body of the device. As such, this would allow the windows to be co-molded with the body 12. Further, if the device 10 is configured as an implantable device, the material used to form the openings 20 also needs to be biocompatible material.

In some embodiments, device 10 may be encased in a pre-formed, sterilized, cover (not shown) held in place by a friction fit over body 12. In embodiments where the device is configured as an external device, the cover may further include a cushioning agent to cushion the device when attached to the patient's body, the cover may also be disposable. The disposable or sterilized nature of the cover ensures that only sterile surfaces contact the patient's body and treatment area. When included, the cover may also have a plurality of openings that are aligned with the (underlying) openings 20 of body 12. In embodiments where the device is configured as an external device, body 12 may further include strap hooks (not shown) for receiving a strap for supporting the device. In other embodiments, the device may be attached to a target area of the patient's body using flexible bandage.

In the depicted example, device 10 includes four light sources 105 distributed over the surface area of the device. The light sources 105 may be configured to generate a low power light. As such, the light sources may be configured to be flexible or non-flexible, based on the flexible nature of the device. As non-limiting examples, the light sources may include any combination of lasers (or laser diodes), light emitting diodes (LEDs), and/or infrared devices. Based on the nature of the light source, the light beam generated by the device may have a wavelength ranging from 400 nm to 1300 nm. In various embodiments, the light beam includes a wavelength in the range of 400 nm to 1300 nm. In various embodiments, the light beam includes a wavelength in the red or near infrared range. In various embodiments, the light beam includes a wavelength in the range of 600 to 900 nm. In various embodiments, the light beam includes a wavelength in the range of 800 to 900 nm. In various embodiments, the light beam includes a wavelength in the range of 840 to 860 nm. In various embodiments, the light beam consists of light having wavelength ranging from 400 nm to 1300 nm. In various embodiments, the light beam consists of light in the red or near infrared range. In various embodiments, the light beam consists of light having wavelength ranging from 600 to 900 nm. In various embodiments, the light beam consists of light having wavelength ranging from 800 to 900 nm. In various embodiments, the light beam consists of light having wavelength ranging from 840 to 860 nm.

In some embodiments, device 10 may further include a focusing lens (not shown) for focusing the low power light generated by the light source onto the body of the patient. However in alternate embodiments, such as where the light source is capable of generating a collimated and focused beam, additional focusing lenses may not be required.

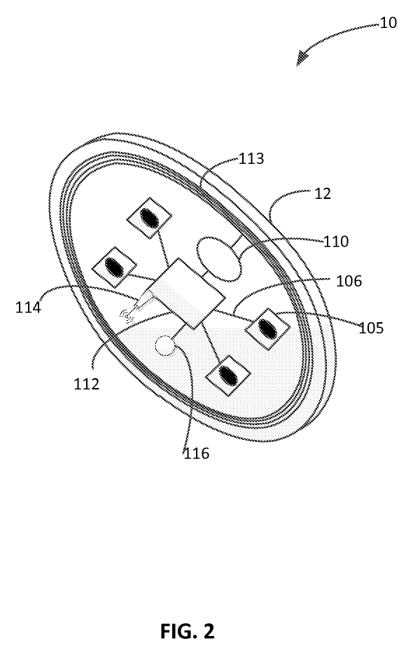

FIG. 2 illustrates the inner electronic components of the light-generating device 10. Light sources 105 may be interconnected inside the device by flat wire connectors 106. The flat wire connectors 106 may lead to a power source 110 and a programmable controller 112. Power source 110 powers the light sources 105. Various small and portable power sources can be used, such as one or more batteries. The batteries may include, for example, one or more wafer thin lithium polymer batteries, or rechargeable nickel-metal hydride batteries. In one example, when rechargeable batteries are included in power source 110, the batteries may be recharged when device 10 is not enabled, or not attached to the patient's body. In various embodiments, a wireless charging coil (113) is included to wirelessly recharge the battery. In this way, the device power source 110 may be recharged wirelessly through an external recharging system while implanted in a patient's body. In various embodiments, an adaptor is included to allow the battery to be charged using an external power source via a wired connection.

Programmable controller 112 may include a control circuit printed onto a circuit board. As such, programmable controller may be configured with code for selectively enabling the light source 105 of the device 10 according to the prescribed light regimen. The prescribed light regimen may be determined by the patient's healthcare provider based on one or more metabolic syndrome parameters of the patient. For example, the light regimen may be based on the patient's triglyceride levels, glucose levels, cholesterol levels, lipoprotein levels and/or ratios (e.g., HDL level, or HDL or LDL ratio), body mass index, body weight etc. The light regimen may include a plurality of emission periods during which low power light is directed towards the specific body part. Each of the plurality of emission periods may be separated from each other by interval periods during which no low power light is generated. As such, the light regimen may be continued until the measured parameters are within or below a threshold range. Example light regimens are discussed below.

Specifically, the programmable controller may selectively enable the light source (for example, by connecting light source 105 to power source 110) during each of the emission periods of the prescribed light regimen, and then selectively disable the light source (for example, by disconnecting light source 105 from power source 110) during each of the interval periods of the prescribed light regimen.

Device 10 may optionally include a wireless receiver (114) for wirelessly communicate with external computing devices. In various embodiments, the wireless receiver is an integral part of the control circuit 112. In various embodiments, computer instructions including for example prescribed light regimen can be communicated to the device 10 from an external computing device via the wireless receiver. The various patient parameters measured through the various sensors can be communicated to an external computing device via the wireless receiver.

In embodiments where the device 10 is configured as an external device, it may optionally include a proximity sensor (not shown) for determining the proximity of the device to the patient's body. The proximity sensor may be configured as a chip included in the body 12 of the device. In one example, the proximity sensor may be activated when the device 10 is within a threshold distance of the patient's body (e.g., when the device is placed on the waist/visceral area of the patient). The proximity sensor may communicate that the sensor (or the device) is within a threshold distance of the patient's body to the healthcare provider (e.g., via wireless communication). In this way, with the help of the proximity sensor, a doctor may be able to monitor the wearing of the device and make sure that the patient is wearing the device as prescribed. In various embodiments, the proximity parameter provided by the proximity sensor may be used to automatically turn on/off the light source. In this way, the light source is turned on only when the device is close to a solid surface (e.g., the body part) to minimize the chance laser light generated by the source does not accidentally cause retinal damage to the patient, a caregiver, or someone standing nearby.

The device may further include one or more patient parameter sensors 116 for measuring one or more patient (e.g., metabolic syndrome) parameters of the patient, such as glucose, cholesterol (HDL & LDL), adiponectin, liver enzyme (SGPT & SGOT), triglyceride, lymphocyte, monocyte, blood pressure, blood oxygen levels, etc. Based on the parameters to be measured, appropriate parameter sensors may be included in the device. In various embodiments, the sensors 116 may include interfaces for measuring the patient parameters. In various embodiments, the patient parameters may be measured via sensors external to the device and communicated to the device via a communication interface.

Since the body 12 of the device houses the light sources, power sources, programmable controller, and various sensors, support posts may be optionally included within body 12 to enhance the rigidity of the device, as well as to provide spacing within the body to delineate the internal components.

In one embodiment (as depicted), light sources 105 are low-power lasers, such as vertical-cavity surface-emitting lasers (VCSELs) having a nominal power output of 2.6 mW and a wavelength in the range of 600-850 nm (herein, a wavelength of approximately 750 nm). The power output of the lasers may range from 2.6 to 10 mW. At this operating level, sufficient power can be provided for the treatment of metabolic syndrome with an extended life of the power source.

As such, VCSELs comprise semiconductor lasers which emit a collimated beam normal to the surface of the semiconductor substance. The semiconductor typically comprises aluminum arsenide (AIAs) or gallium arsenide (GaAs), or a combination thereof. Each VCSEL has a self-contained, high-reflectivity minor structure forming a cavity to produce the collimated beam. Due to the ability of VCSELs to produce a collimated beam, additional focusing lenses may not be required in device to focus the beam when VCSELs are the light source. Additionally, VCSELs provide reduced size and low power consumption benefits. Since typical VCSELs are in the order of 25 micrometers long, and have an operational power threshold below 1 mA, multiple VCSELs can be powered from a single power source.

In one embodiment, the VCSELs are one millimeter or less in height and are embedded in the casing material of body 12. Arrays of VCSELs, spaced by one-half inch, may be “sandwiched” between the polymer materials and interconnected by flat wire connector 106 in the device. The arrays of VCSELs may be electrically connected in parallel to avoid having a single inoperative VCSEL prevent numerous other VCSELs from operating.

In another embodiment, the light source includes one or more light emitting diodes (LEDs), such as hyper-luminescent red (“hyper-red”) LEDs. These LEDs can emit a light beam having 2000-3000 candle power and a wavelength of approximately 850 nm. The hyper-red LEDs may also be arrayed in a manner similar to the VCSELs in device 10. In still other embodiments, a combination of LEDs and VCSELs may be used wherein the hyper-red LEDs and the VCSELS are arrayed in an alternating fashion with several VCSELs for every hyper-red LED.

Operation of the power source 110 and programmable controller 112 may be initiated by means of a switch, such as a single-pole, double-throw or pressure switch. The programmable controller 112 may be configured with code for controlling the operation and timing of light sources 105. As used herein, timing control includes enabling the operation of the light source for the duration of the emission period, and disabling the operation of the light source for the duration of the interval period, in accordance with the prescribed light regimen. The programmable controller may have a 24 hour timing function which adjusts the operation of the light source for the emission and interval periods of time over the course of the 24 hour period.

The programmable controller may further include an oscillator to supply pulses at regular intervals (e.g., at one second intervals) to a counter/timer circuit. The counter circuit counts the pulses while a count decode logic circuit monitors the count. The count decode logic circuit is a multipurpose logic circuit which may comprise, for example, a PAL (programmable array logic) or a PLA (programmable logic array) that may be programmed to detect specified counts based on the emission periods and interval periods of the light regimen. For example, a count of 14,400 which would correspond to four hours of time and a count of 120 which would correspond to two minutes of time. Programmable controller 112 would be capable of maintaining the stored timing program without power being applied thereto. Upon detecting the programmed count, the count decode logic circuit outputs a laser enable pulse which enables laser current regulator circuits which regulate the power to each laser or LED of the light source. The regulator circuits, which are known in the art and which compare the current with a known voltage reference in order to maintain a constant current output, receive a voltage reference input from a voltage reference circuit. The voltage reference circuit may comprise an active band gap zener diode which supplies a constant voltage output (on the order of 1.2 to 1.5 volts) regardless of the voltage of the battery. At the same time, the logic circuit provides a RESET pulse to the counter/timer circuit to reset the count, and the counter continues counting the pulses from the oscillator. The laser enable pulse remains active for the duration of the emission period of the light regimen.

When power source 110 includes a rechargeable battery, the programmable controller 112 may also be configured to connect power source 110 to an external power source to recharge the battery. In some embodiments, power source 110 may be further coupled to a dedicated battery charge controller that is configured to monitor charging of the rechargeable battery and disconnect the battery from the external power source when a threshold charge level is reached.

Power source 110 may be selected so that it is capable of providing sufficient power for a defined duration of the laser therapy (e.g., one day, one week, etc.). A low battery voltage protection circuit may be included in programmable controller 112 to regulate the power supplied by the power source 110 and provide a desired voltage output (e.g., between 3.6 and 4.8 volts). The protection circuit may cease the supply of the power if the battery voltage drops below the threshold level (e.g., 3.6 volts) to avoid damage to circuit components. The power supplied by the power source 110 via the protection circuit is used to power the circuit components as well as the VCSELs and/or hyper-red LEDs. A standard LED driver circuit, as known in the art, may be used to drive the hyper-red LEDs.

In some embodiments, device 10 may include a heat sink coupled to each light source to address excess heat generated by the light source during the device operation. The heat sinks may be formed of copper and can be used to disburse the excess heat produced by the laser diode light source into the surrounding housing material. The heat sinks may also lend added structural integrity to the locations of the laser diodes. However, when VCSELs are used as the light source, the heat sinks may not be required due to low heat generated by the VCSELs.

FIG. 3 illustrates an example external light-generating device with contoured body configured to fit a patient's body contour, such as the body contour of the patient's abdomen area.

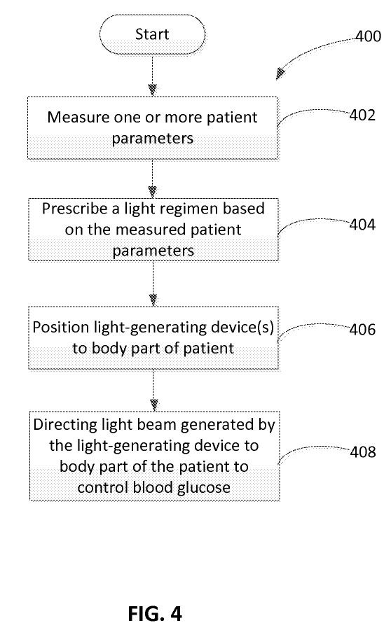

Now turning to FIG. 4, an example method 400 of managing and treating diabetes and pre-diabetes with a light generating device, such as the devices illustrated in FIGS. 1 to 3, is provided. At 402, the method includes measuring one or more patient parameters, such as various metabolic syndrome parameters such as waist circumference (WC), blood pressure, blood glucose, cholesterol, and triglyceride level, and various other patient parameters such as adiponectin level, liver enzyme (SGPT & SGOT) level, and lymphocyte and monocyte counts. A patient parameter may be measured via sensors integral to the light generating device. For example, a blood glucose level may be measured via an onboard glucose sensor integral to the light generating device. Alternatively, a patient parameter may be measured through sensors and processes not integral part of the light generating device. For example, one or more patient parameters may be measured via separate lab tests and the results may be communicated to the light generating devices.

At 404, the method includes prescribing a light regimen. The light regimen may be based on one or more patient parameters such as one or more metabolic syndrome parameters. By adjusting the prescribed light regimen based on measured metabolic syndrome parameters of the patient, such as glucose levels and triglyceride levels, the therapy can be customized for each patient based on the severity of their condition. Specifically, the healthcare provider may program the programmable controller with the details of the prescribed light regimen. In one example, the programmable controller may be preprogrammed with a plurality of light regimens from which the healthcare provider may select an appropriate light regimen depending on the patient's measured metabolic syndrome parameter. In an alternate example, the programmable controller may be provided with a PCMCIA port which can interface with a “smart card” or master programming card which can be inserted to download a desired prescribed light regimen on to the controller by the healthcare provider. As still another example, a serial interface may be provided to connect the light-generating device to a personal computer of the healthcare provider, from where the prescribed light regimen may be downloaded onto the controller.

At 406, the method includes positioning the light-generating device to a target area (e.g., stomach area, thigh area, etc.) of the patient's body.

In various embodiments, the device is attached externally to the body part, such as a body part rich in adipose tissue such as a visceral adipose area of the body (that is, a visceral area with underlying adipose tissue). The device may be a portable device attached to the patient's body part. By using a portable and non-invasive device to provide the laser therapy, the treatment can be undergone without adversely affecting the patient's quality of life. The light-generating device may be attached to the selected part of the patient's body by the healthcare provider (e.g., physician) using a strap or bandage, so as to position the device over the desired treatment location. The strap or bandage may be sterilized, disposable, and made of a stretchable or flexible material (such as nylon) to allow the device to be properly attached to the patient's body while applying a positive biasing force to hold the device in place. Once placed upon the patient's body, the device may essentially look like a “patch”. This improves the wearability of the device. The patch can be worn by the patient for 12 hours per day, or 12 hours at night, and recharged when not worn. Further, since different light regimens can be provided for the patient using the same device (by changing the details on the programmable controller), patients may not need to replace the device frequently. For example, the patient may need a new patch only once every two years.

In various embodiments, the device is implanted internal to the body part, such as a body part rich in adipose tissue such as a visceral adipose area of the body (that is, a visceral area with underlying adipose tissue). In one example, the device is injected into the subcutaneous space of the body part. By using an implanted device implanted, the patient do not have to worry about remembering to wear the device and the treatment can be carried out with assurance of patient compliance.

At 408, the method includes selectively enabling the device according to the prescribed light regimen. As such, operation of the device may be initiated by operating a switch. The light sources of the device may be configured to provide a visible feedback to the user (e.g., by flashing once) to confirm that the device is operational. The healthcare provider may operate the switch upon attaching the device to the patient's body. After the switch is operated and the device is attached to the desired area, the prescribed light regimen can begin.

Optionally, a lag period may be provided between the operation of the switch and the actual initiation of the light regimen to allow sufficient time for the device to be properly positioned on the patient's body prior to initiation of the laser therapy. In embodiments where the device is an external device, the switch may be activated upon the prior activation of the proximity sensor, ensuring that the light regimen is initiated only when the device is placed on the patient's body.

After being positioned to the patient's body, the patient simply wears the device for the prescribed period of time and is free to conduct themselves in the normal course. The device automatically delivers the laser therapy according to the prescribed light regimen, as indicated in the programmable controller, while the patient goes about their normal routine. With such a device, a single doctor visit replaces time consuming and costly, multiple office visits. A physician can initiate the laser treatment regimen using the light-generating device and allow the patient to leave with the device attached to the body for a defined duration, such as one week of laser therapy, without the need for the patient to return to the physician's office. An appointment can be scheduled after that duration for the physician to determine the success of the laser therapy and adjust (or reprogram) the light regimen, for example, based on newly measured metabolic syndrome parameters of the patient. As such, since the device provides precisely timed laser treatments to target areas, the efficiency of the laser treatment is optimized and an overall length of the required treatment can be reduced.

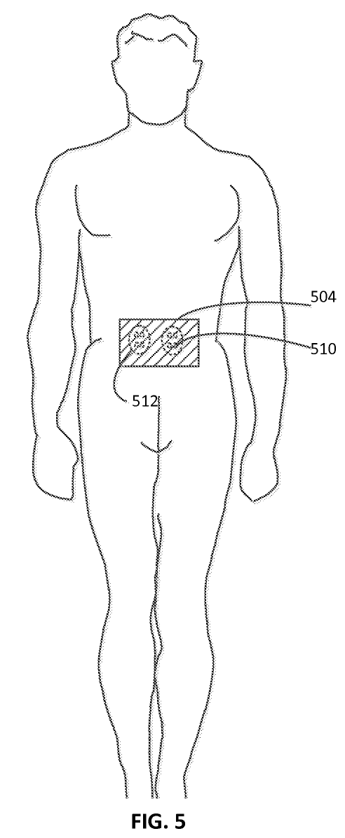

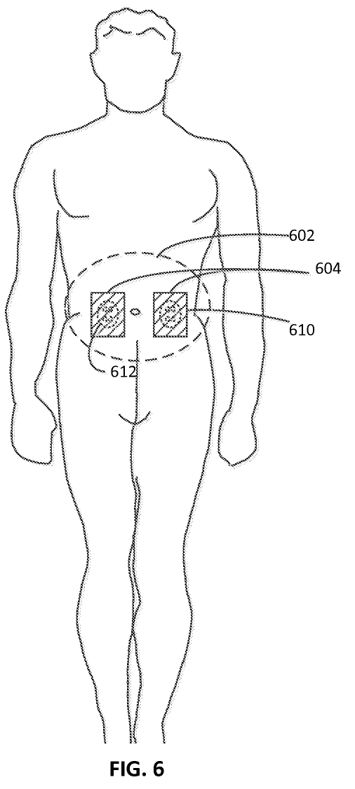

FIGS. 5 and 6 illustrate example embodiments of managing or treating diabetes and metabolic syndrome using a light generating device where the device is attached to a stomach area 302 (or visceral adipose area) of the patient's body.

Specifically, in FIG. 5, a plurality of light-generating devices 510, 512 (herein, two devices) are positioned along a waist circumference, specifically at the level of the navel, to target the low power laser light to the adipose tissue underlying the waist circumference. In the depicted example, each device 510, 512 include four lasers (dotted circles). The devices 510, 512 are kept in place with bandage 504.

In FIG. 6, the plurality of light-generating devices is arranged on the sides of the stomach area 602. Specifically, the plurality of devices are distributed into a left side device 610 that targets the low power laser light on the left side of the stomach area 602, and a right side device 612 that targets the low power laser light on the right side of the stomach area 602. The devices 610, 612 are kept in place with bandage 604.

In both FIGS. 5-6, light beams from the light-generating devices are focused on the patient's visceral adipose tissue. Since the visceral adipose tissue is one of the regions where triglyceride and peptides are stored and released, by focusing the light beams on this target area, the user's sensitivity (e.g., glucose sensitivity) to the light therapy can be increased. As such, the adipose tissue emits triglycerides and causes the endothelial dysfunction, which can lead to cardiovascular disease, kidney failure, and diabetic retinopathy. Thus, by causing an increase in glucose sensitivity, the laser therapy using the light-generating devices may be used to manage the patient's metabolic syndrome by reversing the blood chemistry of individuals with metabolic syndrome, pre-diabetic syndrome, or diabetes.

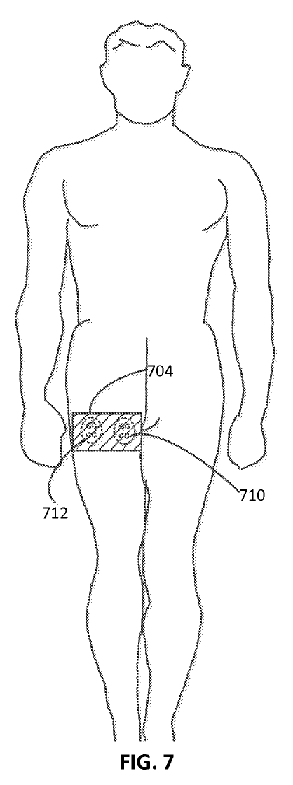

FIG. 7 illustrates another example embodiment for treating or diabetes and metabolic syndrome. Specifically, in FIG. 7, the plurality of light-generating devices 710, 712 are positioned along a front portion of the patient's thigh area 702, using bandage 704, to target the low power laser light to the adipose tissue underlying the thigh and upper leg area. While FIG. 7 shows the plurality of devices aligned along a front portion of the thigh area, it will be appreciated that the devices may be alternatively be positioned on left and right thigh regions, or front and back regions of the thigh area, similar to the positioning shown in FIG. 6.

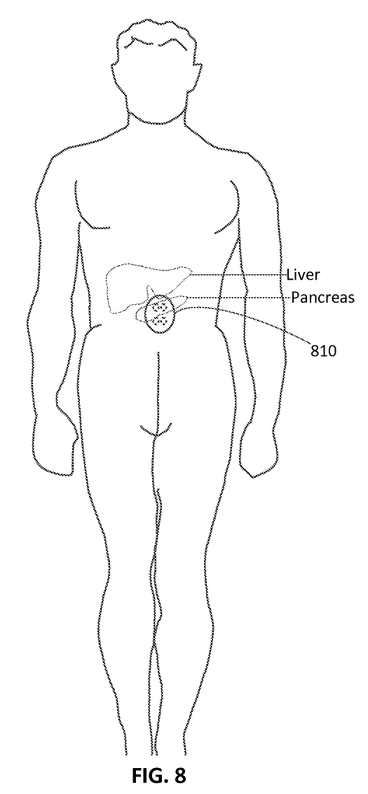

FIG. 8 illustrates an example embodiment of managing or treating diabetes and metabolic syndrome using a light generating device where the device 810 is implanted in the adipose tissue near the pancreas of the patient. The device 810 is oriented in such a way that the light beams generated by the light generating device are directed to the adipose tissue of this area according to a prescribe regimen.

In one example light regimen, the patient may wear two light-generating devices, each device having 4 vertical-cavity surface-emitting laser (VCSEL) 850 nm lasers at the light source that emit light beams over the underlying adipose tissue of the user for 10-14 hours per day. In each device, two of the four lasers may be selectively enabled to dose 9-12 mW of light energy (generated using 15 mA current) on the target area for an emission period of two minutes before being disabled. Next, the other two lasers of the device may be selectively enabled to dose 9-12 mW of light energy on the target area for an emission period of two minutes before being disabled. The lasers of the devices may be selectively disabled for an interval period of sixteen minutes. As such, the emission period followed by the interval period constitutes a cycle which is then repeated over the duration of the regimen. That is, at the end of the sixteen minutes, the lasers may be re-enabled for two minutes durations. In one example, the patient may wear the device for 14 hours during which the cycle (with emission periods separated by interval periods) is constantly repeated. At the end of the 14 hours, the device may be recharged. In one example, where the power source of the device is rechargeable (e.g., lithium ion battery), the power source is recharged for 5 hours after the 14 hour regimen, each day. It should be noted, the above described light regimen is an example light regimen. Other light regimen may be possible within the scope of the invention, for example, other light wavelength may be used, different light intensity may be used to achieve similar results, the emission period and resting period of the light sources may be adjusted, for example emission period may be 1 to 30 minutes and the resting period may be 1 to 60 minutes.

The aforementioned light therapy was tested on 4 patients diagnosed with metabolic syndrome, Type 1 or Type 2 diabetes for various length of time. Various patient parameters were monitored at various points during the study. The results indicate that the light regimen led to normalized blood glucose levels and a reduction in triglyceride levels to within normal levels. Further, adiponectin levels were shown to be within the normal range after treatment, and patient achieved reduction in weight, waist size, and improvement of overall health over time.

Table I shows example results from a patient diagnosed with metabolic syndrome. The patient is a 46 year old female. Patient was tested with a light generating device having 8 lasers. Patient's diet was maintained, with no changes, over the duration of the treatment. Also, no diabetic drugs were administered after initiating of the light therapy. In addition to lowering of glucose and triglyceride levels, the original waist size of the patient was reduced by 2 inches in one month although weight remained the same at the end of the study. Further, adiponectin was in a normal range within 4 months of treatment. Some of the results for the patient are listed below.

TABLE I

| | | 1 Month | | | |
|---|---|---|---|---|---|
| | | Post | 6 Month | 9 Month | 20 Month |
| | Pre-laser | Laser- | Post Laser | Post Laser | Post Laser |
| Blood Test | Treatment | Treatment | Treatment | Treatment | Treatment |
| Fasting | 103 | 94 | 102 | 86 | 86 |
| Glucose | | | | | |
| Cholesterol | 144 | 171 | 166 | 162 | 155 |
| Triglyceride | 215 | 203 | 179 | 169 | 108 |
| HDL | 32 | 35 | 35 | 34 | 35 |
| LDL | 69 | 95 | 95 | 94 | 98 |
| calculated | | | | | |
| Cholesterol/ | 4.5 | 4.9 | 4.7 | 4.8 | 4.4 |
| HDL | | | | | |

Table II shows example results from a patient having Type 2 diabetes. The patient is a 49 year old male. Patient was tested with a light-generating device having 8 lasers. The patient wore the device for 10 hours per day. Prior to treatment with any therapy, the patient's blood glucose level was 468 and the patient was prescribed the diabetic drugs Metformin® and Lantus® to reduce glucose levels. The patient gained 35 lbs during the first month of treatment with Lantus®. After starting treatment with the laser therapy, in addition to stabilizing glucose levels, the original waist size of the patient was reduced by 2.5 inches while weight was reduced by 32 lbs. The patient has been able to manage diabetes without taking Lantus®. The patient was also able to reduce Metformin® usage by half. The patient's blood pressure was returned to with a normal range. Further, adiponectin was in a normal range (7 mcg/l) within 4 months of treatment.

TABLE II

| | Pre-laser | 4 Months Post | 21 Months Post |
|---|---|---|---|
| Blood Test | Treatment | Laser Treatment | Laser-Treatment |
| Fasting Glucose | 94 | 94 | 74-100 |
| Cholesterol | 144 | 171 | 171 |
| Triglyceride | 92 | 133 | 78 |
| HDL | 49 | 49 | 56 |
| Cholesterol/HDL | 2.3 | 2.7 | 2.2 |
| Insulin Injection | 32 u | 10 u | 0 u |
| (Lantus ®) | | | |
| A1C | 5.2 | 5.8 | 5.7 |

Table III shows example results from a second patient having Type 2 diabetes. The patient is a 52 year old male. Patient was tested with a light-generating device having 8 lasers. The patient wore the device for 10 hours per day. Prior to treatment with any therapy, the patient's fasting blood glucose level was 185. After starting treatment with the laser therapy, the patient has lost 18 lbs without changing diet and the patient's fasting glucose is lowered from 185 to 144 in 5 months, to 79 in 12 months. The patient's triglycerides also decreased from 116 to 92 in 7 months. The patient's liver enzyme SGPT decreased from 40 to 19 in 5 months and maintained at 19 at 7 months. The patient's liver enzyme SGOT decreased from 49 to 24 in 5 months and stayed at 30 at 7 months. The decrease in liver enzyme indicates an improvement of liver function. This may be due to reduction of fatty cells in the liver. The patient's VLDL also showed a decrease from 23.2 to 18.4 in 7 months.

TABLE III

| | | 5 Month Post | 7 month Post | 12 Month |
|---|---|---|---|---|
| | Pre Laser | Laser | Laser | Post Laser |
| Blood Test | Treatment | Treatment | Treatment | Treatment |
| GLY HGB-AIC | 7.3 | 7.0 | 6.9 | 6.3 |
| Fasting Glucose | 185 | 144 | 135 | 79 |
| Cholesterol | 131 | 135 | 130 | 130 |
| Triglyceride | 116 | 106 | 92 | — |
| HDL | 37 | 36 | 41 | — |
| VLDL | 23.2 | 21.2 | 18.4 | 18.4 |
| AST/SGOT | 40 | 19 | 19 | — |
| ALT/SGPT | 49 | 24 | 30 | — |

Table IV shows example results of a patient having Type 1 diabetes. The patient is a 29 year old male. Patient was tested with a light-generating device having 8 lasers. The patient wore the device for 10 hours per day. Prior to treatment with any therapy, the patient's fasting blood glucose level was 166, the patient was prescribed insulin injection (Lantus®) 41-45 U in the morning and afternoon to reduce glucose levels and Humalog in the morning. After starting treatment with the laser therapy, the patient has been able to manage diabetes with only morning insulin injection and stopped the Humalog® and is only using Lantus® in the morning and has reduced Lantus® to 40 u from 50 u. The patient's fasting glucose is lowered from 166 to 142 in 5 months, to 85-88 in 9 months. In addition, the weight of the patient was reduced was 2 lbs without change in diet. The patient's lymphocyte decreased from 34.7% to 25.5% in 4 month indicating suppression of immune system. The patient's liver enzyme SGPT decreased from 30 U/L to 18 U/L in 9 months indicating improvement of liver function possibly due to reduction of lipids present in liver. In addition, the patient lost 4 lbs during the study.

TABLE IV

| | | 5 Month Post | 7 month Post | 9 Month |
|---|---|---|---|---|
| | Pre Laser | Laser | Laser | Post Laser |
| Blood Test | Treatment | Treatment | Treatment | Treatment |
| GLY HGB-AIC | 7.6% | 7.3% | — | 7.5% |
| Fasting Glucose | 166 | 142 | 88 | 85 |
| Lymphocyte | 34.7% | 25.5% | — | — |
| Monocyte | 6.1% | 5.5% | — | — |
| ALT (SGPT) | 30 U/L | 30 U/L | — | 18 U/L |
| ALT (SGOT) | 20 U/L | 20 U/L | — | 19 U/L |
| Adiponectin | — | — | 15 mcg/ml | — |
| Insulin Injection | 41-45 u | 41-45 u | 40-43 u | 40-43 u |
| (Lantus ®) | AM & PM | AM & PM | AM | AM |

In summary, the results suggest that by using a low intensity light therapy, diabetes (both Type 1 and Type 2) and metabolic syndrome of a patient may be managed or treated. In particular, by positioning the laser device to an abdomen area having underlying adipose tissue and selectively enabling the laser device according to a prescribed light regimen, glucose levels and triglyceride levels may be reduced. In addition, other metabolic parameters such as liver enzyme and adiponectin level may be normalized, patient weight and waist size may be reduced, and blood pressure normalized

The subject matter of the present disclosure includes all novel and nonobvious combinations and sub-combinations of the various processes, systems, devices, composition of matters, and configurations, and other features, functions, acts and/or properties disclosed herein, as well as any and all equivalents thereof.

The disclosed embodiments and various details are merely illustrative and are not to be construed as limiting and the invention can be implemented in various alternative ways. Further, the scope of the invention is limited only by the claims and the invention encompasses numerous alternatives, modifications and equivalents.

What is claimed is:

1. A method for managing diabetes or pre-diabetes using a light generating device, comprising:
positioning the light generating device to a body part of a patient; and
directing a beam of light generated by the light generating device to the body part of the patient to control blood glucose level of the patient.

2. The method of claim 1, wherein the light beam includes a wavelength of 400 to 1300 nm.

3. The method of claim 1, wherein the light beam is a low intensity beam generated by a power of 1-12 mW.

4. The method of claim 1, wherein the body part includes one body area selected from the group consisting of an adipose tissue rich area: abdominal, thigh, and buttocks.

5. The method of claim 1, wherein the body part is adjacent to the pancreas of the patient.

6. The method of claim 1, wherein positioning the light generating device includes placing the light generating device over the body part but external to the patient body.

7. The method of claim 1, wherein positioning the light generating device includes implanting the light generating device to the body part so that the device is internal to the patient body.

8. The method of claim 1, wherein the light beam is generated by a light emitting diode.

9. The method of claim 1, wherein the light beam is generated by a laser diode.

10. The method of claim 1, wherein light beam is focused by a focusing lens.

11. The method of claim 1, wherein directing the light beam to the body part of the patient is according to a prescribed light regimen.

12. The method of claim 11, wherein the prescribed light regimen is based on one or more patient parameters, wherein one or more of the one or more patient parameters are selected from the group consisting of: patient weight, patient waist size, patient blood sugar level, patient blood lipid level, and patient blood liver enzyme level.

13. The method of claim 11, wherein the light regimen includes a plurality of emission periods during which the low power light is directed towards the body part, each of the plurality of emission periods separated from each other by a resting interval period during which no low power light is generated.

14. The method of claim 1, wherein managing diabetes or pre-diabetes using a light generating device, comprising directing the beam of light generated by the light generating device to the body part of the patient to control one or more selected from the group consisting of: patient weight, patient waist size, blood liver enzyme level, and blood triglyceride level.

15. A light generating device for managing diabetes or pre-diabetes, comprising:
a source configured to generate a light beam;
a controller configured to direct a light beam generated to a body part of a patient to control the blood glucose level of the patient when the light generating device is positioned to the body part of the patient.

16. The device of claim 15, wherein the light beam have wavelength in the range of 400 to 1300 nm.

17. The device of claim 15, wherein the light beam is a low intensity beam generated by a power of 1-12 mW.

18. The device of claim 15, wherein the body part includes on body area selected from the group consisting of an adipose tissue rich area: abdominal, thigh, and buttocks.

19. The device of claim 15, wherein the body part is adjacent to the pancreas of the patient.

20. The device of claim 15, wherein the source includes a light emitting diode for generating the light beam.

21. The device of claim 15, wherein the source includes a laser diode for generating the light beam.

22. The device of claim 15, wherein the light generating device includes a focusing lens for focusing the light beam.

23. The device of claim 15, wherein directing the light beam to the body part of the patient is according to a prescribed light regimen.

24. The device of claim 23, wherein the prescribed light regimen is based on one or more patient parameters.

25. The device of claim 24, wherein one or more of the one or more patient parameters are selected from the group consisting of: patient blood sugar level, patient blood lipid level, and patient blood liver enzyme level.

26. The device of claim 25, wherein the light regimen includes a plurality of emission periods during which the low power light is directed towards the body part, each of the plurality of emission periods separated from each other by a resting interval period during which no low power light is generated.

27. The device of claim 26, wherein each of the plurality of emission periods is 1-4 minutes in length and the resting interval period is 1 to 6 minute in length.

28. The device of claim 15, wherein the light generating device includes a wireless rechargeable battery.

29. The device of claim 15, wherein the light generating device includes one or more sensors for sensing patient parameters.

30. The device of claim 15, wherein the controller is further configured to direct a light beam generated to a body part of a patient to control one or more selected from the group consisting of: patient weight, patient waist size, blood liver enzyme level, and blood triglyceride level.
